(19) United States
(12) Patent Application Publication
(10) Pub. No.: US 2022/0058339 A1
(43) Pub. Date: Feb. 24, 2022

(54) Reinforcement Learning Approach to Modify Sentence Reading Grade Level

(71) Applicant: Michelle Archuleta, Lakewood, CO (US)

(72) Inventor: Michelle Archuleta, Lakewood, CO (US)

(21) Appl. No.: 17/275,951

(22) PCT Filed: Sep. 25, 2019
(86) PCT No.: PCT/US19/53039

§ 371 Date: Mar. 12, 2021

Related U.S. Application Data
(60) Provisional application No. 62/736,148, filed on Sep. 25, 2018.

(51) Int. Cl. G06F 40/253; G06N 20/00; G06F 40/117

(52) U.S. Cl. CPC G06F 40/253; G06F 40/117; G06N 20/00

(57) ABSTRACT

Methods, systems, and apparatus, including computer programs language encoded on a computer storage medium for a language simplification system whereby input jargon language is modified to plain language using a reinforcement learning system with a real-time reward grade level grammar engine. The actions of an agent are to reduce the reading grade level: 1) substituting plain language words for technical terms, 2) splitting long sentences into shorter sentences and rebuilding the sentences to maintain the original meaning. The reinforcement learning agent learns a policy of edits and modifications to a sentence such that the output sentence is grammatical and retains the intended meaning.

### RELATED APPLICATIONS

This application claims priority to U.S. Provisional Patent Application No. 62/736,148 entitled “Reinforcement learning approach to modify sentence reading grade level.” Filed Sep. 25, 2018, the entirety of which is hereby incorporated by reference.

### TECHNICAL FIELD

The present invention relates generally to Artificial Intelligence related to reinforcement learning for grammatical correction. In particular, the present invention is directed to natural language processing and reinforcement learning for simplifying jargon into layman terms and is related to classical approaches in natural language processing such as formal language theory, grammars, and parse trees. In particular, it relates to generalizable reward-mechanisms for reinforcement learning such that the reward mechanism is a property of the environment.

### BACKGROUND ART

There are approximately 877,000 (AAMC The Physicians Foundation 2018 Physician Survey 2018) practicing doctors in the United States. The average number of patients seen per day in 2018 was 20.2 (Id. at pg. 22). The average amount of time doctors spend with patients has decreased to 20 minutes per patient (Christ G. et al. The doctor will see you now—but often not for long 2017). In this limited amount of time physicians are unable to properly explain complex medical conditions, medications, prognosis, diagnosis, and plans for self-care.

Patients' experience of healthcare in the form or written and oral communication is most often incomprehensible due to jargon filled language. Personalized information such as health records, genetics, insurance, etc. while most valuable and pertinent is completely inaccessible to most individuals.

The ability to simplify jargon into plain understandable language can have significant benefits for, e.g., patients. For example, in a medical application, layman language can save lives because a patient that understands their condition, their medication, their prognosis, or their diagnoses will be more likely to be compliant and/or identify medical staff errors.

Manually substituting plain language for medical jargon and rearranging the words such that the sentence makes sense would be a substantial cost to develop for use, e.g., in the healthcare system when healthcare and insurance companies are cutting back. The cost of having doctors simplify EHRs would be unwieldy.

An estimate: 877,000 (total active doctors)×20.2 (patients seen per day)×7.5 (additional minutes for simplifying an EHR note)/1440 (minutes in a day) ˜92,268 additional 24-hr days for the medical workforce per day of seeing patients. The average overall physician salary is $299,000 a year or $143/hour (Kane L, Medscape Physician Compensation Report 2018). Simplifying EHR would result in an additional total cost per year for the entire healthcare system of $4.8B.

The unmet need is to simplify medical jargon into plain language. There are no solutions in the prior art that could fulfill the unmet need of simplifying medical jargon language such as EHRs, insurance, genetics, etc. The prior art is limited by software programs that require human input and human decision points, supervised machine learning algorithms that require massive amounts (10⁹-10¹⁰) of human generated paired labeled training datasets, algorithms that are unable to rearrange words within a sentence to make the sentence understandable, and algorithms that are brittle and unable to perform well on datasets that were not present during training.

### DISCLOSURE OF THE INVENTION

This specification describes a language simplification system that includes a reinforcement learning system and a real-time grade level grammar engine implemented as computer programs one or more computers in one or more locations. The language simplification system components include input data, computer hardware, computer software, and output data that can be viewed by a hardware display media or paper. A hardware display media may include a hardware display screen on a device (computer, tablet, mobile phone), projector, and other types of display media.

Generally, the system performs actions (e.g. splitting and rebuilding sentences) on a sentence using a reinforcement learning system such that an agent learns a policy to perform the actions that reduce the reading grade level while maintaining the grammaticality of the sentence. An environment that is the input sentence, an agent, a state (e.g. word, character, or punctuation), an action (e.g. splitting and rebuilding sentences, simplifying technical terms and/or abbreviations, deletion, insertion, substitution, rearrangement, capitalization, or lowercasing), and a reward (positive—grammatical sentence, positive—reduction in reading grade level negative—non-grammatical sentence, negative—increase in reading grade level) are the components of a reinforcement learning system. The reinforcement learning system is coupled to a real-time grade level grammar engine such that each edit (action) made by an agent to the sentence results in a positive reward if the sentence is grammatical or if there is a reduction in the reading grade level. A negative reward is returned to the agent if the sentence is non-grammatical or has an increase in reading grade level.

In some embodiments real-time grade level grammar engine may be reversed whereby actions are performed (e.g. building longer sentences, substitution with technical terms) to increase the reading complexity of the sentence or sentence(s). In the described embodiment the reinforcement learning system is coupled to a real-time grade level grammar engine such that each edit (action) made by an agent to the sentence results in a positive reward if the sentence is grammatical or if there is an increase in the reading grade level. A negative reward is returned to the agent if the sentence is non-grammatical or has a decrease in the reading grade level.

In general, one or more innovative aspects may be embodied in a generalizable reward mechanism, a real-time grade level grammar engine. A real-time grade level grammar engine when provided with an input sentence, data sources (e.g. grammar, training data), computer hardware including a memory and a processor(s), and a computer program or computer programs when executed by a processor, outputs one of two values that specifies whether a particular sentence is grammatical or non-grammatical.

A generalizable reward mechanism is able to correctly characterize and specify intrinsic properties of any newly encountered environment. The environment of the reinforcement learning system is a sentence. An intrinsic property of a sentence is grammaticality and reading grade level, such that a sentence is or is not well formed in accordance with the productive rules of the grammar of a language. The measure of well formed is such that a sentence complies with the formation rules of a logical system (e.g. grammar).

The intrinsic property of grammaticality is applicable to any newly encountered sentence. In addition, grammaticality is the optimal principal objective for the language simplification system defined in this specification.

A grammar engine builder computer program when executed on a processor or processors builds all of the components to construct a real-time grammar engine for a particular input sentence such that the real-time grammar engine can be immediately executed (‘real-time’) on a processor or processors to determine whether or not the input sentence is grammatical. A reading grade level metric (e.g. Flesch-Kincaid readability test, Flesch Reading Ease, Dale Chall, etc.) is computed before and after an agent performs an action to the sentence.

The grammar engine builder computer program when executed on a processor or processors is provided with a grammar such that the grammar generates a production rule or a plurality of production rules, whereby the production rules describe all possible strings in a given formal language.

The grammar engine builder computer program takes the input sentence and calls another computer program, a part-of-speech classifier, which for every word, character, and/or punctuation the part-of-speech classifier outputs a part-of-speech tag. The grammar engine builder computer program creates a grammar production rule or plurality of grammar production rules by generating the grammar rules that define the part-of-speech tags from the input sentence. The grammar engine builder computer program creates an end-terminal node production rule or plurality of end-terminal node production rules by mapping the part-of-speech tags and the words, character, and/or punctuation in the input sentence to the production rules.

The grammar engine builder computer program is provided with a parser computer program whereby residing on a memory and executed by a processor or processors provide a procedural interpretation of the grammar with respect to the production rules of an input sentence. The parser computer program searches through the space of trees licensed by a grammar to find one that has the required sentence along its terminal branches. The parser computer program provides the output signal upon receiving the input sentence. The output signal provided by the parser in real-time when executed on a processor or processors indicates grammaticality.

The grammar engine builder computer program generates the real-time grammar engine computer program by receiving an input sentence and building a specific instance of grammar production rules that are specific to the part-of-speech tags of the input sentence. The grammar engine builder computer program stitches together the following components: 1) grammar production rule or plurality of grammar production rules, 2) end terminal node production rule or plurality of end terminal node production rules that map to the part-of-speech tags of the input sentence, 3) a grammar parser.

The real-time grammar engine receives the input sentence, and executes the essential components: grammar production rules that have been pre-built for the input sentence, a grammar, and a parser. The real-time grammar engine parses the input sentence and informs a reinforcement learning system that the edits or modifications made by an agent to a sentence result in either a grammatical or non-grammatical sentence.

In some implementations a grammar can be defined as a generative grammar, regular grammar, context free grammar, context-sensitive grammar, or a transformative grammar.

Some of the advantages include a methodology that 1) allows sentences to be evaluated to determine if they are grammatical or not; 2) ungrammatical sentences are corrected using a reinforcement learning algorithm; 3) the neural network implemented in the reinforcement learning algorithm is trained with unparalleled training data derived from extensive language model word embeddings; 4) a reader can personalize the content by specifying a preferred reading grade level.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 illustrates a language simplifications system.

FIG. 2 depicts a reinforcement learning system with example actions.

FIG. 3 illustrates a reinforcement learning system with adjustable targeted reading grade level.

FIG. 4 illustrates a reinforcement learning system with detailed components of the grade level grammar engine.

FIG. 5 depicts a flow diagram for reinforcement learning system with transferrable weights.

### BEST MODE OF CARRYING OUT THE INVENTION

### Language Simplification System

In order to achieve a software program that is able, either fully or partially, to simplify jargon laden sentence into plain language by processing, e.g., electronic health records (EHRs), that program may transform the records into lay person friendly language. The system must overcome the following challenges: 1) rearrange words within a sentence so that the grammar and semantics are preserved; 2) split sentences and rebuild them into shorter simpler sentences, 3) substitute medical words with plain language terms; 4) be able to scale and process large datasets.

Embodiments of the invention are directed to a language simplification system whereby a corpus of jargon filled language is provided by an individual or individuals(s) or system into a computer hardware whereby data sources and the input corpus are stored on a storage medium and then the data sources and input corpus are used as input to a computer program or computer programs which when executed by a processor or processor provides as output plain language which is provided to an individual or individual(s) on a display screen or printed paper.

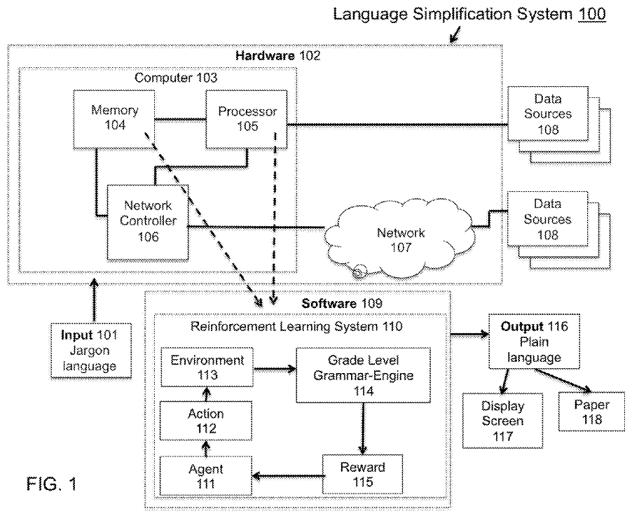

FIG. 1 illustrates a language simplification system 100 with the following components: input 101, hardware 102, software 108, and output 116. The input is jargon language such as language in a EHR, a medical journal, a prescription, a genetic test, and an insurance document, among others. The input 101 may be provided by an individual, individuals or a system and entered into a hardware device 102 such as a computer 103 with a memory 104, processor 105 and or network controller 106. A hardware device is able to access data sources 108 via internal storage or through the network controller 106, which connects to a network 107.

The data sources 108 that are retrieved by a hardware device 102 in one of other possible embodiments includes for example but not limited to: 1) a corpus of medical terms mapped to plain language definitions, 2) a corpus of medical abbreviations and corresponding medical terms, 3) an English grammar that incorporates all grammatical rules in the English language, 4) a corpus of co-occurrence medical words, 5) a corpus of co-occurring words, 6) a corpus of word-embeddings, 7) a corpus of part-of-speech tags.

The data sources 108 and the jargon language input 101 are stored in memory or a memory unit 103 and passed to a software 109 such as computer program or computer programs that executes the instruction set on a processor 105. The software 109 being a computer program executes a reinforcement learning system 110 on a processor 105 such that an agent 111 performs actions 112 on an environment 113, which calls a reinforcement learning reward mechanism, a grade level grammar engine 114, which provides a reward 115 to the system. The reinforcement learning system 110 makes edits to the sentence while ensuring that the edits result in a grammatical sentence at a lower reading grade level. The output 116 from the system is plain language that can be viewed by a reader on a display screen 117 or printed on paper 118.

In one or more embodiments of the language simplification system 100 hardware 102 includes the computer 103 connected to the network 107. The computer 103 is configured with one or more processors 105, a memory or memory unit 104, and one or more network controllers 106. It can be understood that the components of the computer 103 are configured and connected in such a way as to be operational so that an operating system and application programs may reside in a memory or memory unit 104 and may be executed by the processor or processors 105 and data may be transmitted or received via the network controller 106 according to instructions executed by the processor or processor(s) 105. In one embodiment, a data source 108 may be connected directly to the computer 103 and accessible to the processor 105, for example in the case of an imaging sensor, telemetry sensor, or the like. In one embodiment, a data source 108 may be executed by the processor or processor(s) 105 and data may be transmitted or received via the network controller 106 according to instructions executed by the processor or processors 105. In one embodiment, a data source 108 may be connected to the reinforcement learning system 110 remotely via the network 107, for example in the case of media data obtained from the Internet. The configuration of the computer 103 may be that the one or more processors 105, memory 104, or network controllers 106 may physically reside on multiple physical components within the computer 103 or may be integrated into fewer physical components within the computer 103, without departing from the scope of the invention. In one embodiment, a plurality of computers 103 may be configured to execute some or all of the steps listed herein, such that the cumulative steps executed by the plurality of computers are in accordance with the invention.

A physical interface is provided for embodiments described in this specification and includes computer hardware and display hardware (e.g. a printer used for delivering a printed plain language output). Those skilled in the art will appreciate that components described herein include computer hardware and/or executable software which is stored on a computer-readable medium for execution on appropriate computing hardware. The terms “computer-readable medium” or “machine readable medium” should be taken to include a single medium or multiple media that store one or more sets of instructions. The terms “computer-readable medium” or “machine readable medium” shall also be taken to include, but not be limited to, solid-state memories, and optical and magnetic media. For example, “computer-readable medium” or “machine readable medium” may include Compact Disc Read-Only Memory (CD-ROMs), Read-Only Memory (ROMs), Random Access Memory (RAM), and/or Erasable Programmable Read-Only Memory (EPROM). The terms “computer-readable medium” or “machine readable medium” shall also be taken to include any non-transitory storage medium that is capable of storing, encoding or carrying a set of instructions for execution by a machine and that cause a machine to perform any one or more of the methodologies described herein. In other embodiments, some of these operations might be performed by specific hardware components that contain hardwired logic. Those operations might alternatively be performed by any combination of programmable computer components and fixed hardware circuit components.

In one or more embodiments of the language simplification system 100 software 109 includes the reinforcement learning system 110 which will be described in detail in the following section.

In one or more embodiments of the language simplification system 100 the output 116 includes layman friendly language. An example would be layman friendly health records which would included: 1) modified grammatical simplified sentences, 2) original sentences that could not be simplified or edited but are tagged for visual representation. The output 116 of layman friendly language will be delivered to an end user via a display medium such as but not limited to a display screen 117 (e.g. tablet, mobile phone, computer screen) and/or paper 118.

Additional embodiments may be used to further the experience of a user such as the case of health records. An intermediate step may be added to language simplification system 100 such that the plain language 116 is output in a display screen 117 that can then be reviewed by an expert, edited by an expert, and addition comments from the expert saved with the plain language 116. An example is a simplified health record that is reviewed by a doctor. The doctor also is able edit a sentence and provides a comment with further clarification for a patient. The doctor is then able to save edits and comments and then submit the plain language 116 health record to her patient's electronic health portal. The patient would received the plain language 116 health record and view it on the display screen of his tablet after logging into his patient portal.

### Reinforcement Learning System

Further embodiments are directed to a reinforcement learning system that performs actions to simplify sentence or sentences whereby, a real-time grade level grammar-engine reward mechanism returns a reward that is dependent on the grammaticality and reading grade level of the sentence. The embodiment of a reinforcement learning system with a real-time grade level grammar-engine reward mechanism enables actions such as but not limited to: 1) splitting sentence and rebuilding into simplified sentences; 2) substituting plain language words for technical medical terms; 3) reordering word phrases within a sentence to make the sentence understandable.

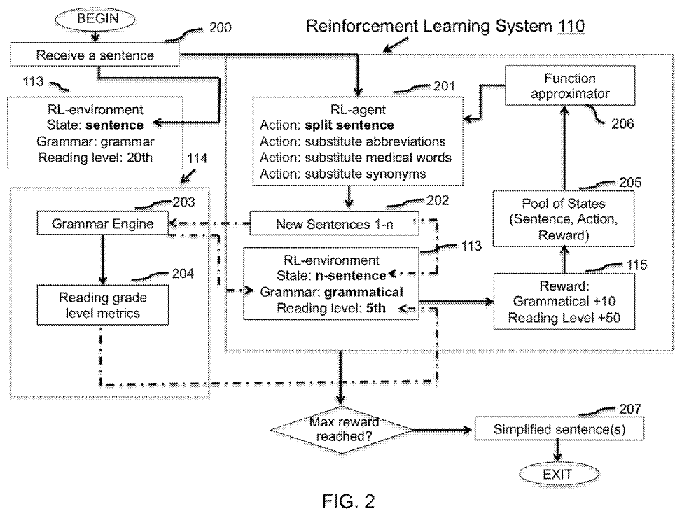

A reinforcement learning system 110 with grade level grammar-engine reward mechanism is defined by an input 101, hardware 102, software 108, and output 207. FIG. 2. illustrates an input to the reinforcement learning system 110 that may include but is not limited to a sentence 200 that is preprocessed and either modified or unmodified by another computer program or computer programs from the input jargon language 101. Another input includes data sources 108 that are provide to the grade-level grammar engine 114 and function approximator 206 and will be described in the following sections.

The reinforcement learning system 110 uses a hardware 102, which consists of a memory or memory unit 104, and processor 105 such that software 109, a computer program or computer programs is executed on a processor 105 and performs edits to the sentence resulting in grammatical simplified sentences 207. The output from reinforcement learning system 110 in an embodiment is combined in the same order as the original jargon language such that the original language is reconstructed to produce plain language output 116. A user is able to view the plain language output 116 on a display screen 117 or printed paper 118.

FIG. 2 depicts a reinforcement learning system 110 with an input sentence 200 and an environment that holds state information consisting of: the sentence(s), the grammaticality of the sentence(s) 113, and reading grade level of the sentence(s); such that an agent performs actions 112 (example actions 201); and a grade-level grammar engine 114 is used as the reward mechanism returning a positive reward 115 if the sentence has a lower reading grade level and is grammatical, and a negative reward if the sentence is non-grammatical or has no reduction in reading grade level 115. Detailed components of the grade-level grammar engine 114 are shown in FIG. 2, which includes a grammar engine 203 and a reading grade level metric 204.

An agent receiving the sentence is able to perform example actions 201 (e.g. splitting sentences, substitution, rearrangement etc.) on the sentence resulting in a new sentence 201. The new sentence(s) 202 is updated in the environment and then passed to a grade-level grammar engine 114 which updates the environment with a grammar state (True-grammatical sentence, False-non-grammatical sentence) and reading grade level. The grade-level grammar engine 114 also returns a reward 115 to the reinforcement-learning environment such that the following rewards are given: 1) a change resulting in a grammatical sentence results in a positive reward; 2) change resulting in a reduction in the reading grade level results in a positive reward; 3) a change resulting in a non-grammatical sentence results in a negative reward; 4) and/or no reduction in reading grade level or an increase in reading grade level results in a negative reward.

A pool of states 205 saves the state (e.g. sentence), action (e.g. splitting sentences), reward (e.g. positive). After exploration and generating a large pool of states 205 a function approximator 206 is used to predict an action that will result in the greatest total future reward. The reinforcement learning system 110 is thus learning a policy to perform edits to a sentence resulting in a grammatically correct sentences at a lower reading grade level. One or more embodiments specify termination once a maximum reward is reached and returns a grammatically simplified sentence 204. Additional embodiments may have alternative termination criteria such as termination upon executing a certain number of iterations among others. Also for given input sentences 200 it may not be possible to produce a grammatically correct sentence or a sentence with a lower reading grade level 207 in such instances the original sentence could be returned and highlighted such that an end user could differentiate between simplified sentence and original jargon language.

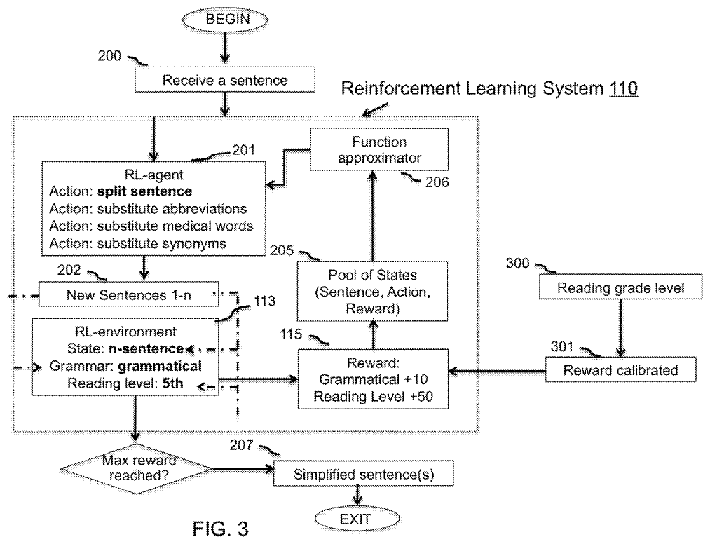

FIG. 3 illustrates a reinforcement learning system 110 with a grade-level grammar engine 114 and an adjustable reading grade level 300 whereby a reward is calibrated 301 to return a positive reward for the reduction of the reading grade level set as a user specified input 300. The reinforcement learning system is optimizing a policy such that it returns simplified sentences at the user defined reading grade level. An advantage of this embodiment is that text can be personalized to a reader's own individual reading grade level.

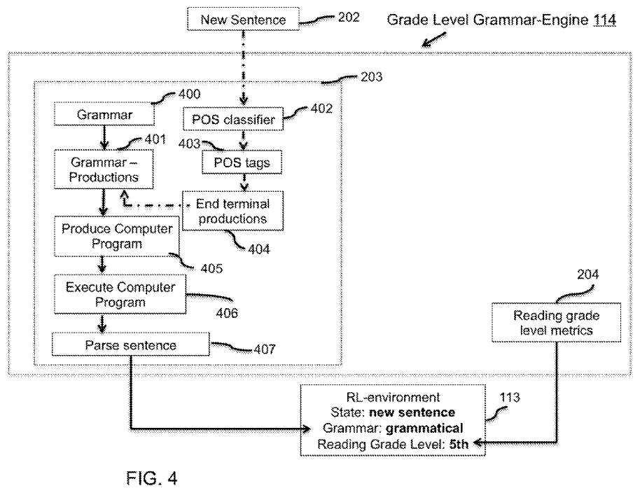

FIG. 4 illustrates a reinforcement learning system 110 with detailed components of the grade-level grammar engine 114. The grade-level grammar engine 114 as shown in FIG. 2 has a grammar-engine 203 and a reading grade level metric 204. FIG. 4 shows additional components of the grammar engine 114. A grammar 400 is defined and used as an input data source 104 such that grammatical productions 401 are produced for the input sentence. A part-of-speech (POS) classifier 402 is used to determine the part-of-speech for each word, character, or punctuation in the sentence such that a POS tag 403 is returned. The POS tags 403 are then used to produce end terminal productions 404 for the corresponding grammar 400 that relates to the new sentence 202. The final grammar productions 401 and a parser are written to a computer program 405. The computer program stored in memory 104 receives a new sentence 202 and executes on a processor the computer program 405 such that the new sentence 202 is parsed. The output of the grade-level grammar engine 114 is both an executable computer program 406 and the value that specifies whether the sentence was grammatical or non-grammatical. A corresponding positive reward 115 is given for a grammatical sentence and a negative reward 115 is given for a non-grammatical sentence.

FIG. 4 illustrates a reading grade level metric 204 which may include the following methods among others: 1) Flesch-Kincaid readability test, 2) Flesch Reading Ease, 3) Dale Chall Readability, 4) Automated Readability Index, 5) Coleman Liau Index, 6) Gunning Fog, 7) SMOG, and 8) Linear Write. In addition a user could provide a customized reading grade level metric.

In some aspects an alternative metric could be substituted with the reading grade level such as a native speaker metric, sentence length metric, word length metric, common word metric, among others.

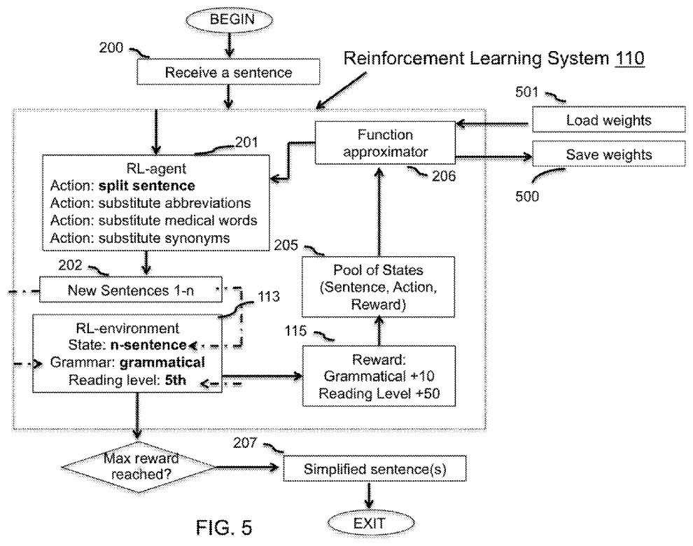

FIG. 5 illustrates a reinforcement learning system 110 with transferrable learning mechanism. The transferrable learning mechanism is weights from a function approximator (e.g. convolutional neural network CNN) that has optimized a learning policy whereby a minimal number of edits that result in a grammatical sentence have been learned. The weights from a function approximator 206 can be stored in a memory 104 such that the weights are saved 500. The weights can be retrieved by a reinforcement learning system 110 and loaded into a function approximator 501. The transferrable learning mechanism enables the optimal policy from a reinforcement learning system 110 to be transferred to a naive reinforcement learning system 110 such that the system 110 will have a reduction in the amount of time required to learn the optimized policy.

### Substitutions Technical Terms & Abbreviations

The technical and abbreviation substitution method uses a hardware 102, which consists of a memory or memory unit 104, and processor 105 such that the method, a computer program or computer programs is executed on a processor 105 and substitutes plain language for abbreviations and/or technical terms resulting in modified sentences 202. The reinforcement learning system 110 uses the technical and abbreviation substitution method whereby an agent selects an action (e.g. technical and abbreviation substitution method) and receives a reward if the action resulted in a reduction in reading grade level and/or resulted in a grammatical sentence.

The following example illustrates one of many possible embodiments for the technical and abbreviation substitution method. The first step is to filter the words in the sentence with stop words, which increases the overall efficiency of the method. The second step is to load a multi-level dictionary for technical work substitution or abbreviation diction for abbreviation substitution into memory 302 and indexed the dictionary for search optimization. If an exact match is found insert the plain language term removing the medical term; otherwise if multiple partial matches are found select the plain language term to insert for the longest medical word partial match.

In some cases there may be ambiguity in the dictionary such that an abbreviation maps to more than one medical word. For example the abbreviation IDA′ matches with two possible medical words ‘low dose aspirin’ and ‘left anterior descending’. In a particular embodiment a bi-directional recurrent neural network (biRNN) is trained on abbreviations, long-form words, and sentences containing a long-form words, which is replaced with the abbreviation. The biRNN is used to predict the long-form word from the context of the sentence that contains the abbreviation.

In another embodiment an deep-learning attention mechanism is used predict the long-form word from the context of the sentence that contains the abbreviation. A deep-learning attention mechanism uses a vector of importance weights in order to predict or infer a word (e.g. abbreviation) in the sentence. The attention vector ‘attends to’ a target abbreviation and/or word within the sentence whereby other words, characters, and/or punctuation within the sentence are correlated with the target abbreviation and/or word. This and other methods are used to resolve abbreviation and or medical word ambiguity.

### Splitting & Rebuilding Sentences

A splitting and rebuilding sentences method uses a hardware 102, which consists of a memory or memory unit 104, and processor 105 such that the method, a computer program or computer programs is executed on a processor 105 and splits sentences and rebuilds sentences into shorter sentences resulting in modified sentences 202. The reinforcement learning system 110 uses the splitting and rebuilding sentences method whereby an agent selects an action (e.g. splitting and rebuilding sentences method) and receives a reward if the action resulted in a reduction in reading grade level and/or resulted in a grammatical sentence.

The following example illustrates one of many possible embodiments for the splitting and rebuilding sentence method. The first step is to characterize a sentence to find the natural separators. The second step is to filter the list of natural separators and remove hanging separators. A hanging separator would result in fragments that could not be rebuilt into complete sentences. The third step is to filter out key word separators (e.g. ‘is when’ and ‘which’). The fourth step is to select a separator or separators, with preference given to separators that are more evenly distributed in the sentences. The fourth step is to split the sentence.

The next embodiment of the invention rebuilds the sentence with the objective to retain the intended meaning of the original sentence with a set of shorter sentences. A part-of-speech (POS) classifier is used to predict the POS tags for all words, characters, numbers, and/or punctuation in the original sentence and the split sentence fragments. A set of features is extracted from the words and POS tags in the original sentence and sentence fragments. Example of features may include but are not limited to: n-grams with words, POS phrases, location of POS tags in sentences, co-occurrence words, word embeddings, character embeddings, among others.

In some embodiments a machine learning method (ML) (e.g. decision tree, naïve bayes, etc) can be trained on input data whereby the ML predicts the noun and verb given the original sentences, and sentence fragments such that the noun and verb are used to rebuild sentence fragments that do not have a noun or a verb.

In some embodiments a reinforcement learning (RL) agent can be used to rebuild the sentence whereby the real-time grade level grammar engine provides a positive reward for complete grammatical sentences and a negative reward for a sentence fragment.

In some embodiments an attention mechanism can be used to predict the noun and verbs that ‘attends’ a particular fragment in the original sentence. The predictive noun and verb attention vector is then used to rebuild the sentence.

In some embodiments a deep learning biRNN can be used to predict the noun and verbs that shares codependency with the sentence fragments. The predictive noun and verb with the highest predictive codependency is then used to rebuild the sentence.

### Operation of Reinforcement Learning System

One of the embodiments provides the grade level grammar-engine reward such that a sentence can be evaluated in real-time and a set of actions performed on a sentence that does not parse in order to restore the grammatical structure while at the same time reducing the reading grade level of the sentence. In this embodiment a sentence and thus its attributes (e.g. grammar, reading grade level) represents the environment. An agent can interact with a sentence and receive a reward such that the environment and agent represent a Markov Decision Process (MDP). The MDP is a discrete time stochastic process such that at each time step the MDP represents some state s, (e.g. word, character, number, and/or punctuation) and the agent may choose any action a that is available in state s. The action is constrained to include all members belonging to a state group. The process responds at the next time step by randomly moving into a new state s′2 and passing new state s′2 residing in memory to a real-time grade level grammar engine that when executed on a processor returns a corresponding reward Rₐ (s,s2) for s′2.

The benefits of this and other embodiments include the ability to personalize reading material to the appropriate reading grade level and the ability to evaluate and correct a sentence in real-time. This embodiment has application in many areas of natural language processing in which a sentence maybe modified and then evaluated for its structural integrity. These applications may include sentence simplification, machine translation, sentence generation, and text summarization among others. These and other benefits of one or more aspects will become apparent from consideration of the ensuing description.

One of the embodiments provides an agent with a set of words within a sentence or a complete sentence and attributes of which include a model and actions, which can be taken by the agent. The agent is initialized with number of features per word, 128, which is the standard recommendation. The agent is initialized with max words per sentence 20, which is used as an upper limit to constrain the search space. The agent is initialized with a starting index within the input sentence.

The agent is initialized with a set of hyperparameters, which includes epsilon ε (ε=1), epsilon decay, ε_decay (ε_decay=0.999), gamma, γ (γ=0.99), and a loss rate η (η=0.001). The hyperparameter epsilon ε is used to encourage the agent to explore random actions. The hyperparameter epsilon ε, specifies an ε-greedy policy whereby both greedy actions with an estimated greatest action value and non-greedy actions with an unknown action value are sampled. When a selected random number, r is less than epsilon ε, a random action a is selected. After each episode epsilon ε is decayed by a factor ε_decay. As the time progresses epsilon ε, becomes less and as a result fewer non-greedy actions are sampled.

The hyperparameter gamma, γ is the discount factor per future reward. The objective of an agent is to find and exploit (control) an optimal action-value function that provides the greatest return of total reward. The standard assumption is that future rewards should be discounted by a factor γ per time step.

The final parameter the loss rate, η is used to reduce the learning rate over time for the stochastic gradient descent optimizer. The stochastic gradient descent optimizer is used to train the convolutional neural network through back propagation. The benefits of the loss rate are to increase performance and reduce training time. Using a loss rate, large changes are made at the beginning of the training procedure when larger learning rate values are used and decreasing the learning rate such that a smaller rate and smaller training updates are made to weights later in the training procedure.

The model is used as a function approximator to estimate the action-value function, q-value. A convolutional neural network is the best mode of use. However, any other model maybe substituted with the convolutional neural network (CNN), (e.g. recurrent neural network (RNN), logistic regression model, etc.).

Non-linear function approximators, such as neural networks with weight θ make up a Q-network which can be trained by minimizing a sequence of loss functions, Lᵢ(θᵢ) that change at each iteration i,

Lᵢ(θᵢ)=E_{s,a˜ρ(⋅)}[(yᵢ−Q(s,a;θ)²)

where

$${y}_{i} = {E}_{s , a ∼ ρ ( \cdot ) ; ∼ ξ} ⌈ ( r + {γmax}_{} Q ( ; {Θ}_{i - 1} ) \vert s , a ) ⌉ \qquad$$

is the target for iteration i and ρ(s, a) is a probability distribution over states s or in this embodiment sentences s. and actions a such that it represents a sentence-action distribution. The parameters from the previous iteration θᵢ are held fixed when optimizing the loss function, Lᵢ(θᵢ). Unlike the fixed targets used in supervised learning, the targets of a neural network depend on the network weights. Taking the derivative of the loss function with respect to the weights yields,

$${∇}_{{Θ}_{i}} {L}_{i} ( {Θ}_{i} ) = {E}_{( s , a ∼ ρ ( \cdot ) ;} ⌈ ( r + γ {max}_{} Q ( ; {Θ}_{i - 1} ) - Q ( s , a ; {Θ}_{i} ) ) {∇}_{{Θ}_{i}} Q ( s , a ; {Θ}_{i} ) ⌉$$

It is computationally prohibitive to compute the full expectation in the above gradient; instead it is best to optimize the loss function by stochastic gradient descent. The Q-learning algorithm is implemented with the weights being updated after an episode, and the expectations are replaced by single samples from the sentence-action distribution, ρ(s, a) and the emulator ξ.

The algorithm is model-free which means that is does not construct an estimate of the emulator ξ but rather solves the reinforcement-learning task directly using samples from the emulator ξ. It is also off-policy meaning that it follows ε-greedy policy which ensures adequate exploration of the state space while learning about the greedy policy a=maxₐQ (s, a; θ).

A CNN was configured with a convolutional layer equal to the product of the number of features per word and the maximum words per sentence, a filter of 2, and a kernel size of 2. The filters specify the dimensionality of the output space. The kernel size specifies the length of the 1D convolutional window. One-dimensional max pooling with a pool size of 2 was used for the max-pooling layer of the CNN. The model used the piecewise Huber loss function and adaptive learning rate optimizer, RMSprop with the loss rate, η hyperparameter.

After the model is initialized as an attribute of the agent, a set of actions are defined that could be taken for each word within an operational window in the sentence. The model is off-policy such that it randomly selects an action when the random number, r [0,1] is less than hyperparameter epsilon ε. It selects the optimal policy and returns the argmax of the q-value when the random number, r [0,1] is greater than the hyperparameter epsilon ε. After each episode epsilon ε is decayed by a factor ε_decay, a module is defined to decay epsilon ε. Finally, a module is defined to take a vector of word embeddings and fit a model to the word embeddings using a target value.

One of the embodiments provides a way in which to map a sentence to its word-embedding vector. Word embedding comes from language modeling in which feature learning techniques map words to vectors of real numbers. Word embedding allows words with similar meaning to have similar representation in a lower dimensional space. Converting words to word embeddings is a necessary pre-processing step in order to apply machine learning algorithms which will be described in the accompanying drawings and descriptions. A language model is used to train a large language corpus of text in order to generate word embeddings.

Approaches to generate word embeddings include frequency-based embeddings and prediction based embeddings. Popular approaches for prediction-based embeddings are the CBOW (Continuous Bag of Words) and skip-gram model which are part of the word2vec gensim python packages. The CBOW in the word2vec python package on the Wikipedia language corpus was used.

A sentence is mapped to its word-embedding vector. First a large language corpus (e.g. English Wikipedia 20180601) is trained on the word2vec language model to generate corresponding word embeddings for each word. Word embeddings were loaded into memory with a corresponding dictionary that maps words to word embeddings. The number of features per word was set equal to 128 which is the recommended standard. A numeric representation of a sentence was initialized by generating a range of indices from 0 to the product of the number of features per word and the max words per sentence. Finally a vector of word embeddings for an input sentence is returned to the user.

One of the embodiments provides an environment with a current state, which is the current sentence that may or may not have been modified by the agent. The environment is also provided with the POS-tagged current sentence and a reset state that restores the sentence to its original version before the agent performed actions. The environment is initialized with a maximum number of words per sentence.

One of the embodiments provides a method for measuring a reading grade level both before and after an agent has performed an action. Examples of methods that could be used but are not limited to include the following: 1) Flesch-Kincaid readability test, 2) Flesch Reading Ease, 3) Dale Chall Readability, 4) Automated Readability Index, 5) Coleman Liau Index, 6) Gunning Fog, 7) SMOG, and 8) Linear Write. In addition a user could provide a customized reading grade level metric.

One of the embodiments provides a reward module that returns a negative reward r− if the sentence length is equal to zero; it returns a positive reward r+ if a grammar built from the sentence is able to parse the sentence; it returns a positive reward r+ if a reduction in reading grade level occurs; returns a negative reward r− if a grammar built from the sentence is unable to parse the sentence; returns a negative reward r− if sentence had an increase in reading grade level.

At operation, a sentence is provided as input to a reinforcement-learning algorithm a grammar is generated in real-time from the sentence. The reading grade level is computed for the sentence. The sentence, grammar, and reading grade level represents an environment. An agent is allowed to interact with the sentence and receive the reward. In the present embodiment, at operation the agent is incentivized to perform actions to the sentence that result in grammatically correct sentences at a reduced reading grade level.

One of the embodiments provides a reward module that returns a negative reward r− if the sentence length is equal to zero; it returns a positive reward r+ if a grammar built from the sentence is able to parse the sentence; it returns a positive reward r+ if a addition in reading grade level occurs; returns a negative reward r− if a grammar built from the sentence is unable to parse the sentence; returns a negative reward r− if sentence had an decrease in reading grade level.

At operation in the alternative embodiment, a sentence is provided as input to a reinforcement-learning algorithm a grammar is generated in real-time from the sentence. The reading grade level is computed for the sentence. The sentence, grammar, and reading grade level represents an environment. An agent is allowed to interact with the sentence and receive the reward. In the alternative embodiment, at operation the agent is incentivized to perform actions to the sentence that result in grammatically correct sentences and an increase in reading grade level.

First a min size, batch size, number of episodes, and number of operations are initialized in the algorithm. The algorithm then iterates over each episode from the total number of episodes; for each episode e, the sentence s, is reset from the environment reset module to the original sentence that was the input to the algorithm. The algorithm then iterates over k total number of operations; for each operation the sentence s is passed to the agent module act. A number, r is randomly selected between 0 and 1, such that if r is less than epsilon e, the total number of actions, nₜₒₜₐₗ is defined such that nₜₒₜₐₗ=nₐ^{w}^{ₛ} where nₐ is the number of actions and wₛ is the words in sentence s. An action a, is randomly selected between a range of 0 and nₜₒₜₐₗ and the action a, is returned from the agent module act.

After an action a, is returned it is passed to the environment. Based on the action a, a vector of subactions or a binary list of 0s and 1s for the length of the sentence s is generated. After selecting subactions for each word in a sentence s the agent generates a new sentence s2 from executing each subaction on each word in sentence s.

The binary list of 0s and 1s may include the action of deleting words if the indexed word has a ‘1’ or keeping words if the indexed word has a ‘0’. The sentence s2 is then returned and passed to the reward module.

A grammar is generated for the sentence s2 creating a computer program for which the sentence s2 is evaluated. If the grammar parses the sentence a positive reward r+ is returned otherwise a negative reward r− is returned. If k, which is iterating through the number of operations is less than the total number of operations a flag terminate is set to False otherwise set flag terminate to True. For each iteration k, append the sentence s, before action a, the reward r, the sentence s2 after action a, and the flag terminate to the tuple list pool. If k<number of operations repeat previous steps else call the agent module decay epsilon, e by the epsilon decay function e_decay.

Epsilon e is decayed by the epsilon decay function e_decay and epsilon e is returned. If the length of the list of tuples pool is less than the min size repeat steps previous steps again. Otherwise randomize a batch from the pool. Then for each index in the batch set the target=r, equal to the reward r for the batch at that index; generate the word embedding vector s2_vec for each word in sentence 2, s2 and word embedding vector s_vec for each word in sentence, s. Next make model prediction X using the word embedding vector s_vec. If the terminate flag is set to False make model prediction X₂ using the word embedding vector s2_vec. Using the model prediction X₂ compute the q-value using the Bellman equation: q−value=r+γ max X₂ and then set the target to the q-value. If the terminate flag is set to True call agent module learn and pass s_vec and target and then fit the model to the target.

The CNN is trained with weights θ to minimize the sequence of loss functions, Lᵢ(θᵢ) either using the target as the reward or the target as the q-value derived from Bellman equation. A greedy action a, is selected when the random number r is greater than epsilon e. The word embedding vector s_vec is returned for the sentence s and the model then predicts X using the word embedding vector s_vec and sets the q-value to X. An action is then selected as the argmax of the q-value and action a returned.

Reinforcement Learning does not Require Paired Datasets.

The benefits of a reinforcement learning system 110 vs. supervised learning are that it does not require large paired training datasets (e.g. on the order of 10⁹ to 10¹⁰ (Goodfellow I., 2014)). Reinforcement learning is a type of on-policy machine learning that balances between exploration and exploitation. Exploration is testing new things that have not been tried before to see if this leads to an improvement in the total reward. Exploitation is trying things that have worked best in the past. Supervised learning approaches are purely exploitative and only learn from retrospective paired datasets.

Supervised learning is retrospective machine learning that occurs after a collective set of known outcomes is determined. The collective set of known outcomes is referred to as paired training dataset such that a set of features is mapped to a known label. The cost of acquiring paired training datasets is substantial. For example, IBM's Canadian Hansaard corpus with a size of 10⁹ cost an estimated $100 million dollars (Brown, 1990).

In addition, supervised learning approaches are often brittle such that the performance degrades with datasets that were not present in the training data. The only solution is often reacquisition of paired datasets which can be as costly as acquiring the original paired datasets.

### Real-Time Grade Level Grammar Engine

One or more aspects includes a real-time grade level grammar engine, which consists of a shallow parser and a grammar, such as, but not limited to, a context free grammar, which is used to evaluate the grammar of the sentence and return a reward or a penalty to the agent. A real-time grade level grammar engine is defined by an input (101, 201), hardware 102, software 108, and output (113 & 115). A real-time grade level grammar engine at operation is defined with an input sentence 201 that has been modified by a reinforcement learning system 110, a software 109 or computer program that is executed on hardware 102 that includes a memory 104 and a processor 105 resulting in an output a value that specifies a grammatical sentence vs. a non-grammatical sentence. The output value updates the reinforcement learning system environment (113) and provides a reward (115) to the agent (111).

One or more aspects of a context free grammar, as defined in formal language theory, is a certain type of formal grammar such that sets of production rules describe all possible strings in a given formal language. These rules can be applied regardless of context. A formal language theory deals with the hierarchies of language families defined in a wide variety of ways and is purely concerned with the syntactical aspects rather than the semantics of words. They can also be applied in reverse to check whether a string is grammatically correct. These rules may include all grammatical rules that are specified in any given language.

One or more aspects of a parser processes input sentences according to the productions of a grammar, and builds one or more constituent structures that conform to the grammar. A parser is a procedural interpretation of the grammar. The grammar is a declarative specification of well-formedness such that when a parser evaluates a sentence against a grammar it searches through the space of trees licensed by a grammar to find one that has the required sentence along its terminal branches. If a parser fails to return a match the sentence is deemed non-grammatical and if a parser returns a match the sentence is said to be grammatical.

An advantage of a grade level grammar engine is that it has sustained performance in new environments. An example is that the grade level grammar engine can correct a sentence from doctor's notes and another sentence from a legal contract. The reason being that grade level grammar engine rewards an agent based on whether or not a sentence parses. The grammaticality of the sentence is a general property of either a sentence from a doctor's note or a sentence in a legal contract. In essence in selecting a reward function, the limited constraint introduced in the aspect of the reinforcement learning grammar-engine was the design decision of selecting a reward function whose properties are general to new environments.

A reinforcement learning system updates a policy such that modifications made to a sentence are optimized to a grammatical search space. A grammatical search space is generalizable and scalable to any unknown sentence that a reinforcement learning system may encounter.

A real-time grade level grammar engine in operation, which receives a sentence 201, and then outputs a computer program with grammar rules that when executed on a processor 105 return the grammaticality of the input sentence 201. First the input sentence 201 is parsed to generate a set of grammar rules. A parse tree is generated from the sentence; the sentence is received 201 from the reinforcement learning environment 110; each word in the sentence is tagged with a part-of-speech tag 403; a grammar rule with the start key S that defines a noun, verb, and punctuation is defined 401; a shallow parser grammar is defined, such as a grammar that chunks everything as noun phrases except for verbs and prepositional phrases; the shallow parser grammar is evaluated using a parser, such as nitk.RegexpParser; and parse the part-of-speech tagged sentence using the shallow parser.

After parsing the sentence a set of grammar rules are defined. The grammar rules start with the first rule that includes the start key S that defines a noun, verb, and punctuation; a grammar rule is initialized for each part-of-speech tag in the sentence; then for each segment in the parse tree a production is appended to the value corresponding part-of-speech keys for the grammar rules; additional atomic features for each individual grammar tags, such as singularity and plurality of nouns, are added to the grammar rules; all intermediate production are produced, such as PP→IN NP; finally, for each word in the sentence a production is created which corresponds to the words POS tag and appends a new grammar rule (e.g. NNS→dogs).

After creating a set of grammar rules and productions the grammar rules are written to a computer program stored on a memory 104, which is then used to evaluate the grammaticality of the sentence by executing the computer program on a processor 105. The computer program is executed on a processor 105; and if the sentence parses return value True otherwise value False. The value is returned to the reinforcement learning system 110 such that a positive reward 115 is returned if the sentence parse returns a True and a negative reward 115 is returned if the sentence parse returns False.

In some implementations a grammar, a set of structural rules governing the composition of clauses, phrases, and words in a natural language maybe defined as a generative grammar whereby the grammar is a system of rules that generates exactly those combinations of words that form grammatical sentences in a given language. A type of generative grammar, a context free grammar, specifies a set of production rules describe all possible strings in a given formal language. Production rules are simple replacements and all production rules are one-to-one, one-to-many, or one-to-none. These rules are applied regardless of context.

In some implementations a grammar maybe defined as a regular grammar whereby a formal grammar is right-regular or left-regular. A regular grammar has a direct one-to-one correspondence between the rules of a strictly right regular grammar and those of a nondeterministic finite automaton, such that the grammar generates exactly the language the automaton accepts. All regular grammars generate exactly all regular languages.

In some implementations a grammar maybe defined as a context-sensitive grammar such that the syntax of natural language where it is often the case that a word may or may not be appropriate in a certain place depending on the context. In a context-sensitive grammar the left-hand sides and right-hand sides of any production rules may be surrounded by a context of terminal and nonterminal symbols.

In some implementations a grammar maybe defined as a transformative grammar (e.g. grammar transformations) such that a system of language analysis recognizes the relationship among the various elements of a sentence and among the possible sentences of a language and uses processes or rules called transformations to express these relationships. The concept of transformative grammars is based on considering each sentence in a language as having two levels of representation: a deep structure and a surface structure. The deep structure is the core semantic relations of a sentence and is an abstract representation that identified the ways a sentence can be analyzed and interpreted. The surface structure is the outward sentence. Transformative grammars involve two types of production rules: 1) phrase structure rules 2) transformational rules such rules that convert statements to questions or active to passive voice, which acted on the phrase markers to produce other grammatically correct sentences.

### Generalizable Reward Mechanism Performs Well in New Environments.

Reinforcement learning with traditional reward mechanism does not perform well with new environments. An advantage of one or more embodiments of the reinforcement learning system described in this specification is that the real-time grade level grammar engine reward mechanism represents a generalizable reward mechanism or generalizable reward function. A generalizable reward mechanism, generalizable function, is able to correctly characterize and specify intrinsic properties of any newly encountered environment. The environment of the reinforcement learning system is a sentence.

The intrinsic property of grammaticality is applicable to any newly encountered environment (e.g. sentence or sentences). An example of different environments is a corpus of health records vs. a corpus of legal documents. The different environments may be different linguistic characteristics of one individual writer vs. another individual writer (e.g. Emergency Room (ER) physician writes in shorthand vs. a general physician who writes in longhand).

From the description above, a number of advantages of some embodiments of the reinforcement learning grade level grammar-engine become evident:

(a) The reinforcement learning grade level grammar-engine is unconventional in that it represents a combination of limitations that are not well-understood, routine, or conventional activity in the field as it combines limitations from independent fields of natural language processing and reinforcement learning.

(b) The grade level grammar engine can deliver personalized content such that the content is tailored to an individuals reading grade level.

(c) The grade level grammar engine can be considered a generalizable reward mechanism in reinforcement learning. An aspect of the grade level grammar engine is that a grammar is defined in formal language theory such that sets of production rules or productions of a grammar describe all possible strings in a given formal language. The limitation of using a grammar defined by formal language theory enables generalization across any new environment, which is represented as a sentence in MDP.

(d) An advantage of the reinforcement learning grammar-engine is that it provides significant costs savings in comparison to supervised learning either traditional machine learning or deep learning methods. The acquisition cost of paired datasets for a 1 million word multi-lingual corpus are $100 k-$250 k. The cost savings comes from applying reinforcement learning, which is not limited by the requirement of paired training data.

(e) An advantage of the reinforcement learning grammar-engine is that it scalable and can process large datasets creating significant cost savings. The calculation provided in the Background section for manually simplifying doctor's notes into patient friendly language shows that such an activity would cost the entire healthcare system $4.8B per year in USD.

(f) Several advantages of the reinforcement learning grammar-engine applied to simplifying doctors notes into patient friendly language are the following: reduction of healthcare utilization, a reduction in morbidity and mortality, a reduction in medication errors, a reduction in 30-day readmission rates, an improvement in medication adherence, an improvement in patient satisfaction, an improvement in trust between patients and doctors and additional unforeseeable benefits.

### INDUSTRIAL APPLICABILITY

A language simplification system could be applied to the following use cases in the medical field:

1) A patient receives a medical pamphlet in an email from his doctor on a new medication that he will be taking. There are medical terms in the pamphlet that are unfamiliar to him The patient using a tablet could copy and paste the content of the medical pamphlet into the language simplification system, select his preferred reading grade level, and hit the submit button. The simplification system would retrieve a storage medium and execute a computer program(s) on a processor(s) and return the content of the medical pamphlet simplified into plain language, which would be displayed for the patient on a display screen on his iPad.

2) A doctor enters a patient's office visit record into the EHR system and clicks on a third-party application containing the simplification system and the input patient record. The doctor then clicks the simplify button. The simplification system would retrieve a storage medium and execute a computer program(s) on a processor(s) and return the content of the patient's office visit record simplified into plain language and customized to a reading grade level which would be reviewed by a doctor using the display screen of her workstation. After the doctor completed her review the doctor then forwards the simplified patient note to the patient's electronic healthcare portal. The patient can view the note is his patient portal using the display screen of his Android phone.

3) A patient is diagnosed with melanoma and wants to understand the latest clinical trial for a drug that was recently suggested by her oncologist. The findings of the clinical trial were published in a peer-reviewed medical journal but she is unable to make sense of the paper. She copies the paper into the language simplification system, selects a preferred reading grade level and hits the simplify button. The simplification system would retrieve a storage medium and execute a computer program(s) on a processor(s) and return the content of the peer-reviewed medical journal into plain language, which she can view, on the display of her iPad.

Other specialty fields that could benefit from a language simplification system include: legal, finance, engineering, information technology, science, arts & music, and any other field that uses jargon.

1. A reinforcement learning system, comprising:
one or more processors; and
one or more programs residing on a memory and executable by the
one or more processors, the one or more programs configured to:
receive a sentence; perform actions on the sentence; select an action to maximize an expected future value of a reward function; and, wherein the reward function depends on: reducing the reading grade level while maintaining the grammaticality of the sentence.

2. The system of claim 1, wherein the reward function is a grade level grammar engine.

3. The system of claim 2, wherein grade level grammar engine returns a positive reward if the action resulted in a grammatical sentence.

4. The system of claim 2, wherein grade level grammar engine returns a positive reward if the action resulted in a reduction in reading grade level.

5. The system of claim 2, wherein grade level grammar engine returns a negative reward if the action resulted in a non-grammatical sentence.

6. The system of claim 2, wherein grade level grammar engine returns a negative reward if the action resulted in an increase in reading grade level.

7. The system of claim 2, wherein the grade level grammar engine consists of a parser that processes the sentences according to the productions of a grammar, wherein the grammar is a declarative specification of well formed, and the parser executes a sentence stored in memory against a grammar stored in memory on a processor and returns the state of the sentence as grammatical or non-grammatical.

8. The system of claim 7, wherein the grade level grammar engine is using a grammar defined in formal language theory such that sets of production rules describe all possible strings in a given formal language.

9. The system of claim 8, wherein the grade level grammar engine can be used to describe all or a subset of rules for any language or all languages or a subset of languages or a single language.

10. The system of claim 9, wherein the grade level grammar engine uses a context free grammar.

11. The system of claim 9, wherein the grade level grammar engine uses a context sensitive grammar.

12. The system of claim 9, wherein the grade level grammar engine uses a regular grammar.

13. The system of claim 9, wherein the grade level grammar engine uses a generative grammar.

14. The system of claim 9, wherein the grade level grammar engine uses transformative grammar such that a Deep structure is changed in some restricted way to result in a Surface Structure.

15. The system of claim 7, wherein the grade level grammar engine is executed on a processor in by first executing a part-of-speech classifier on words and punctuation belonging to the input sentence stored in memory on a processor generating part-of-speech tags stored in memory for the input sentence.

16. The system of claim 15, wherein the grade level grammar engine is executed on a processor by creating a production or plurality of productions that map the part-of-speech tags stored in memory to grammatical rules which are defined by a selected grammar stored in memory.

17. A method for reinforcement learning, comprising the steps of:
receiving one or more sentences;
selecting an action to maximize the expected future value of a reward function; wherein the reward function depends on at least partly on: reducing the reading grade level while maintaining the grammaticality of the sentence.

18. The method of claim 17, wherein the reward function is a grade level grammar engine.

19. The method of claim 18, wherein grade level grammar engine returns a positive reward if the action resulted in a grammatical sentence.

20. The method of claim 18, wherein grade level grammar engine returns a positive reward if the action resulted in a reduction in reading grade level.

21. The method of claim 18, wherein grade level grammar engine returns a negative reward if the action resulted in a non-grammatical sentence.

22. The method of claim 18, wherein grade level grammar engine returns a negative reward if the action resulted in an increase in reading grade level.

23. A reinforcement learning system, comprising:
one or more processors; and
one or more programs residing on a memory and executable by the
one or more processors, the one or more programs configured to:
receive a sentence; perform actions on the sentence; select an action to maximize an expected future value of a reward function; and, wherein the reward function depends on: increasing the reading grade level while maintaining the grammaticality of the sentence.

24. The system of claim 23, wherein the reward function is a grade level grammar engine.

25. The system of claim 24, wherein grade level grammar engine returns a positive reward if the action resulted in a grammatical sentence.

26. The system of claim 24, wherein grade level grammar engine returns a positive reward if the action resulted in an increase in the reading grade level.

27. The system of claim 24, wherein grade level grammar engine returns a negative reward if the action resulted in a non-grammatical sentence.

28. The system of claim 24, wherein grade level grammar engine returns a negative reward if the action resulted in a reduction in the reading grade level.
